(19) United States
(12) Patent Application Publication
(10) Pub. No.: US 2011/0065084 A1
(43) Pub. Date: Mar. 17, 2011

(54) INTEGRATED OXYGEN MEASUREMENT AND CONTROL FOR STATIC CULTURE VESSELS

(76) Inventors: Govind Rao, Columbia, MD (US); Yordan Kostov, Columbia, MD (US)

(21) Appl. No.: 12/991,947

(22) PCT Filed: May 12, 2009
(86) PCT No.: PCT/US09/43540

§ 371 Date: Nov. 10, 2010

Related U.S. Application Data
(60) Provisional application No. 61/127,343, filed on May 12, 2008.

(51) Int. Cl. C12Q 3/00; C12M 1/36

(52) U.S. Cl. 435/3; 435/286.7

(57) ABSTRACT

A system and method for measuring and controlling oxygen in a culture vessel utilizes a non-invasive oxygen sensor and an agitator. A controller controls the agitator based on feedback supplied by the oxygen sensor. The agitator is used to increase oxygen transport into the liquid phase thereby raising the level of oxygen in the culture medium. The agitator driven equilibration results in precise control of the culture medium oxygen.

This application claims priority to U.S. Provisional Application Ser. No. 61/127,343, filed May 12, 2008, whose entire disclosure is incorporated herein by reference.

### BACKGROUND OF THE INVENTION

1. Field of the Invention

The present invention relates to the sensing and control of bioprocess parameters and, more particularly, to non-invasive, integrated sensing and control of oxygen in static culture vessels.

2. Background of the Related Art

Bioprocesses are important in a wide variety of industries such as pharmaceutical, food, ecology and water treatment, as well as to ventures such as the human genome project (Arroyo, M. et al., Biotechnol. Prog. 16: 368-371 (2000); Bakoyianis, V. and Koutinas, A. A., Biotechnol. Bioeng. 49: 197-203 (1996); Bylund, F. et al, Biotechnol. Bioeng. 69: 119-128 (2000); Handa-Corrigan, A. et al., J. Chem. Technol. Biotechnol. 71: 51-56 (1998); López-López, A. et al., Biotechnol. Bioeng. 63: 79-86 (1999); McIntyre, J. J. et al., Biotechnol. Bioeng. 62: 576-582 (1999); Pressman, J. G. et al., Biotechnol. Bioeng. 62: 681-692 (1999); Yang, J.-D. et al., Biotechnol. Bioeng. 69: 74-82 (2000)).

In particular, stem cell research provides an increasingly important path to treating many human diseases. To fully realize the benefits of the research, large quantities of regenerative material will be needed. These quantities can only be obtained by effective and efficient in vitro cultivation, using means which most closely simulate in vivo growth. In vivo, stem cells live in specialized niches and pO₂ is critical in determining their growth and differentiation.

To simulate such growth in vitro, techniques are required which accurately monitor and control pO₂ in the vessels used in the cultivation of stem cells. To date, the most common technique is to manipulate pO₂ externally by varying gas phase levels of oxygen in an incubator gas supply under the assumption that pO₂ in the culture vessels will track the external supply. However, this assumption may not be accurate, in that there is a need for more precise control and measurement of the oxygen level in the medium, thereby allowing growth conditions in vitro to more closely match the normal physiological stem cell environment.

### SUMMARY OF THE INVENTION

An object of the invention is to solve at least the above problems and/or disadvantages and to provide at least the advantages described hereinafter.

Therefore, an object of the present invention is to provide a system and method for non-invasively monitoring and independently controlling, in real time, pO₂ levels around the cells in individual culture vessels, such as T-flasks.

To achieve at least the above objects, in whole or in part, there is provided a system that includes a culture vessel for holding a culture medium, an oxygen sensor positioned inside the culture vessel for detecting dissolved oxygen in the culture medium, wherein the oxygen sensor is adapted to be monitored non-invasively, an agitator attached to the culture vessel for providing agitation to the culture medium, and a controller for determining a dissolved oxygen content of the culture medium based on data from the oxygen sensor and for controlling the agitator based on the dissolved oxygen content of the culture medium.

To achieve at least the above objects, in whole or in part, there is also provided a system that includes an incubator, at least two culture vessels positioned inside the incubator for holding respective culture media, an oxygen sensor positioned in each of the at least two culture vessels for detecting dissolved oxygen in each culture vessel, wherein the oxygen sensors are adapted to be monitored non-invasively, an agitator attached to each of the at least two culture vessels for providing agitation to each culture medium, and a controller for determining a dissolved oxygen content of the culture medium in each culture vessel based on data from the oxygen sensors and for independently controlling each agitator based on the dissolved oxygen content of the respective culture medium.

To achieve at least the above objects, in whole or in part, there is also provided a method that includes non-invasively monitoring dissolved oxygen levels in at least two culture media, and selectively and independently agitating the at least two culture media based on the dissolved oxygen levels in each culture medium.

To achieve at least the above objects, in whole or in part, there is also provided a method that includes non-invasively monitoring dissolved oxygen levels in a culture medium, and selectively agitating the culture medium based on the dissolved oxygen levels the culture medium.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1 are plots illustrating several hypothetical profiles that can be achieved by varying the gas environment in an incubator;

FIG. 2 is a schematic diagram showing the principles of operation of an oxygen sensor patch used in the present invention;

FIG. 3 is a plot showing gas phase and liquid phase medium oxygen measurements in hybridomas;

FIG. 4 is a plot showing gas phase and liquid phase medium oxygen measurements in hybridomas while the T-flask was being agitated, in accordance with the present invention;

FIG. 5 is a schematic diagram of a system for non-invasive measurement and control of pO₂ in a culture vessel, in accordance with one embodiment of the present invention;

FIG. 6 is a plot showing oxygen control in three wells of a 24 well plate using agitation of each well, in accordance with the present invention;

FIG. 7 is shows an oxygen sensor module and an ADC used in one preferred embodiment of the present invention;

FIG. 8 shows a vibration motor used in one preferred embodiment of the present invention;

FIGS. 9A-9D illustrate examples of other types of agitators that may be used to agitate the culture medium, in accordance other embodiments of the present invention; and

FIG. 10 is a schematic diagram illustrating how the system for non-invasive measurement and control of pO₂ in a culture vessel can be placed inside an incubator, in accordance with the present invention.

### DETAILED DESCRIPTION OF PREFERRED EMBODIMENTS

By way of example, the present invention will be described in connection with the monitoring and control of pO₂ levels in a stem cell cultivation environment. However, it should be appreciated that the present invention can be used to monitor and control pO₂ levels in any type of static culture vessel.

The description below cites numerous technical references, which are listed in the Appendix below. The numbers shown in parenthesis at the end of some of the sentences refer to specific references listed in the Appendix. For example, a “(1)” shown at the end of a sentence refers to reference “1” in the Appendix below. All of the references listed in the Appendix below are incorporated by reference herein in their entirety.

Adult stem cells have attracted enormous attention due to their promise as an unlimited source of regenerative material to treat a large number of human disease conditions (1-8). In particular, the multilineage potential of adult cells is attractive as it sidesteps controversies related to embryonic stem cells (43).

While many advances have been made in our ability to isolate adult stem cells, their culture, differentiation and expansion remains more art than science. It is believed that in order to fully understand and control stem cell behavior, the in vivo niches that they originate from need to be replicated in vitro (9-11). Recently, it has become evident that the culture conditions and, in particular, the oxygen level under which the cells are grown result in varying phenotypes (12-15). Thus, it is critical to control the oxygen level in in vivo stem cell cultures.

It has been assumed that the concentration of gaseous O₂ supplied into the incubator is the same that is achieved in the liquid medium in which cells grow. However, this critical premise is inaccurate in the case of both static and shaken culture vessels, and the oxygen levels that the cells experience in the liquid medium are quite different than the gas concentration.

In the description that follows, 100% oxygen is referred to as equivalent to air saturation. This stems from the bioprocessing practice of calibrating oxygen sensors to read 0% in equilibrium with nitrogen and 100% in equilibrium with air. All literature data referred to henceforth has been converted to these units. Therefore, a reviewer going back to a cited paper may find that what we refer to as 100% is referred to as 21% (or in some cases as 20% when 5% CO₂ is present) in the original citation. It is also important to understand that the statement % oxygen refers to equilibrium between the gas and liquid phase. Therefore, 100% oxygen concentration (or level or pO₂) means that the gas phase is at 21% oxygen and is in equilibrium with the liquid. Note that the actual oxygen available to the cells in absolute terms is much lower and is determined by the solubility of oxygen in the medium, which is approximately 8 mg/l or a mere 250 μM in distilled water at 35° C. In practice, solutes decrease this even more and this is why oxygen supply to cells growing in liquids is such a challenge.

Every one of these studies cited below where oxygen levels are referred to used gas phase equilibration to achieve the stated oxygen level in the medium. However, the actual oxygen level experienced by the cells has NOT been continuously measured. Nevertheless, as the studies show, growth and differentiation are significantly affected by pO₂, and thus its accurate measurement and control are critical.

Hyperoxia has been long known to have deleterious effects on all types of cells due to oxidative stress caused by increased oxygen free radical production (33). More recently, hypoxia also appears to play a critical role in cell migration, growth and regulation largely via induction of Hypoxia Inducible Factor (HIF) (28, 34, 35). Hypoxia's role in stem cells is also drawing more scrutiny and appears to be important both in normal as well as in cancer stem cells (12-15, 36).

Stem cells are regarded to occupy niches, which have been compared to ecological niches (9, 11). It would appear that oxygen plays a major role in this niche, as recent evidence points to the stem cells residing in a hypoxic environment (12, 13). It has been shown that when some cells are grown under hypoxic (10-15%) compared to normoxic (100%) conditions, they proliferated more and displayed phenotypic changes. In addition, differentiation ability has been found to be significantly affected. For example, Malladi et al. show that adipose derived MSCs have severely diminished osteogenesis capability when cultured at 10% oxygen versus 100% (37).

Different types of cells have been grown in the presence of low oxygen. For example maintenance of cord blood progenitor cells under low oxygen condition was comparable to growing them on irradiated stromal cells (15). Adult and Neonatal fibroblasts seeded as single cells did not proliferate under 100% oxygen but had been shown to expand and form colonies when grown under low oxygen culture condition (38). In the case of CD34+ bone marrow cells, low oxygen culture condition increased their proliferation rate significantly and increased the number of colony forming units (39). Murine mesenchymal stem cells (MSCs) under hypoxic culture conditions showed greater migration and higher secretion of VEGF and tube formation (40).

It has been demonstrated that culturing rat neural stem cells in more physiological oxygen condition (10-15% O2) increases cell proliferation and enhances clonal expansion and reduces apoptosis (41). These studies also showed an effect of oxygen level in culture on differentiation of NSPCs. It was observed that lowered oxygen enhanced dopaminergic neuronal phenotype upon differentiation (41).

In another study, ASCs were suspended in alginate beads and cultured in control or chondrogenic media in either hypoxic (25%) or normoxic oxygen tension (100%) for up to 14 days (42). Under chondrogenic conditions, low oxygen tension greatly inhibited the proliferation of ASC cells, but was found to induce a two-fold increase in the rate of protein synthesis and a three-fold increase in total collagen synthesis. Hypoxia was found to increase glycosaminoglycan synthesis at certain timepoints. These findings suggest oxygen tension plays an important role in regulating the proliferation and metabolism of ASC cells as they undergo chondrogenesis, and provide evidence that the exogenous control of oxygen tension may provide a means of increasing the overall accumulation of matrix macromolecules in tissue-engineered cartilage (42).

Ultimately, if stem cells are to ever become successful in therapy, their in vivo niches need to be recreated in vitro (11). As the brief literature survey presented above indicates, oxygen levels are a critical factor in stem cell physiology and it is important to measure and control these levels. A recent report has raised the possibility of using hypoxia to arrest stem cell differentiation (12). This report may give rise to a strategy of expanding stem cells under controlled hypoxia before the induction of normoxic differentiation. For such a strategy to be implemented, oxygen levels would need to be reliably measured and controlled under in vitro culture conditions.

As their therapeutic use increases, stem cells will need to be manufactured under Food and Drug Administration (FDA) oversight and licensing. A critical aspect of obtaining approval for therapeutic use will require that the manufacturing conditions follow Good Manufacturing practices (GMP). The FDA is actively encouraging industry to implement Process Analytical Technologies (PAT) as a part of this effort in order to obtain consistent product quality. As a parallel, one can look at the biopharmaceutical industry where protein-based therapeutics are typically produced by mammalian cells grown in bioreactors. In these systems, oxygen, pH and temperature levels are controlled in order to achieve consistent scale-up and product quality as measured by amino acid sequence, glycosylation profiles, purity and potency. Similar metrics for stem cells will also be required and the present invention can help validate the degree to which oxygen level control can be used to make a consistent product as measured by markers and differentiation assays.

Given the clear evidence that oxygen levels impact the fate and proliferation of cells, vendors of incubators have made available systems which allow the investigator to blend gas mixtures to achieve desired pO₂ levels in culture. It has been believed that the cells in the incubator quickly achieve equilibrium with the gas environment. This premise has been used in the references cited above to achieve hypoxic and normoxic conditions. Based on the observed results, it appears that elaborate conditioning protocols may be effective in preparing cells for different applications, such as expansion, preconditioning for implantation, differentiation, wound priming etc..

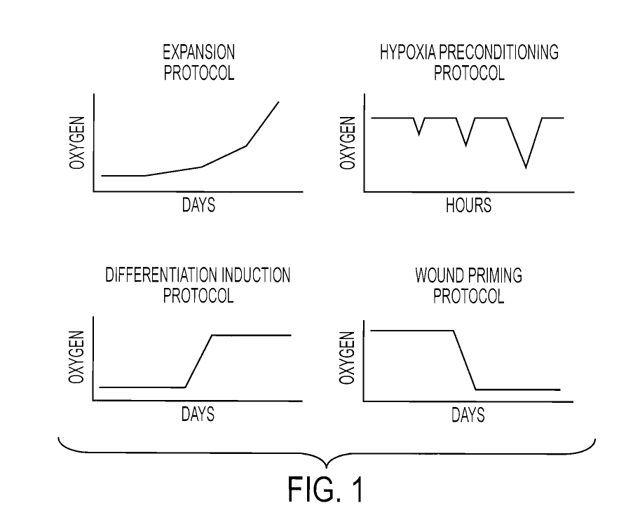

FIG. 1 illustrates several hypothetical profiles that can be achieved by varying the gas environment in the incubator. Again, the critical assumption underlying all these approaches is that the cells growing in liquid media are in equilibrium with the gas phase. We have tested this basic and key assumption because, if it is correct, protocols that are currently in use are appropriate. However, if it is not correct, a major rethinking of how cells are cultured becomes necessary.

The present invention allows for feedback control for individual T-flasks, which is not achievable with prior art shaker incubators. One could achieve quasi-control by manipulating the rocking rate of a shaker incubator, but shaker incubators do not provide the means to achieve feedback control by using the output of an oxygen sensor that measures the liquid phase pO₂. In order to truly replicate an in vivo stem cell niche, the present invention controls pO₂ based on feedback from an actual liquid pO₂. The problem with currently published studies where oxygen levels have been manipulated using the gas phase is that the precise oxygen levels experienced by the cells are not known in those studies. The cells would have been normoxic or hypoxic, but the exact degree to which they were and the reproducibility of the experiments are questionable without actual measurements.

Commonly assigned and related U.S. Pat. Nos. 7,041,493 and 6,673,532, both by Govind Rao at al., describe a system and method for non-invasively measuring cultivation parameters in culture vessels. One of the parameters that can be measured is dissolved oxygen.

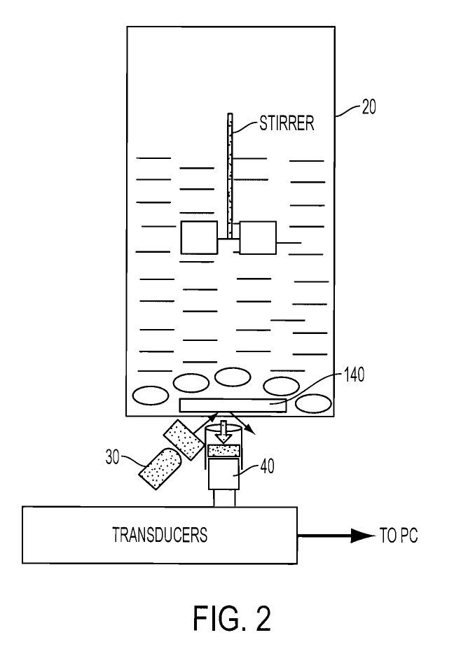

The optical oxygen sensors work on equilibrium principles. FIG. 2 illustrates their principle of operation. A sterilizable oxygen sensor patch 140 with chemistry unique to oxygen is affixed inside the vessel 20 where measurements are to be made. The oxygen sensor patch 140 is illuminated at an appropriate wavelength from the outside using, for example, an LED 30 and the resulting fluorescence signal is measured using a photodetector 40. The oxygen concentration in the vessel is deduced from a previously made calibration curve. The major advantages of this approach are as follow:
In situ measurement—no sampling needed
Non-invasive—no penetration into the vessel thus avoiding possible contamination
Low-cost light sources, semiconductor detectors can be used
Measures through the material (any transparent vessel)
Simple calibration—patches are pre-calibrated
Miniaturization is possible

Other technologies, such as blood gas analyzers, are available. However, their main drawback is the necessity of a sample from the culture vessel, which is inconvenient and labor intensive. Other approaches require invasive insertion of electrodes/optrodes into the culture vessel, requiring modification of the culture vessel and increasing the risk of contamination. The electrodes also require individual calibration, precluding their use in large numbers of culture vessels.

In order to determine whether cells that are growing in liquid media in static culture flasks in an incubator are in equilibrium with the gas phase, an experiment was performed where hybridoma cells were cultured in a CO₂ incubator where the headspace gas concentration was 5% CO₂ and the balance was air. A T-flask was set up with two oxygen sensor patches inside it. One of the oxygen sensors was located in the headspace above the liquid and the other one was located in the liquid (44). Continuous measurements of oxygen were made for the duration of the culture, with samples withdrawn once a day to determine cell count.

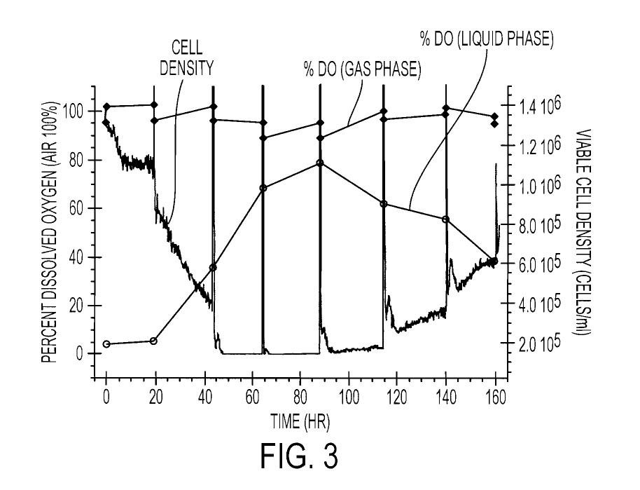

As can be seen in the plot of FIG. 3, the headspace oxygen level remained close to 100%, indicating that it was in equilibrium with the incubator atmosphere. The liquid oxygen level shows a very different profile. As the cells grew (indicated by the viable cell counts), the oxygen consumption rate became greater than the oxygen diffusion rate into the medium (as evidenced by the decreasing oxygen level in the liquid phase) until it reached zero. The cells stayed in this hypoxic regime and the liquid phase oxygen levels only started increasing gradually as the viable cell counts started decreasing as the cells started dying. The spikes in oxygen levels were caused by the mixing of the flask contents that took place when a sample was withdrawn. However, the increased oxygen was temporary as it was quickly consumed once the flasks were static again.

These data clearly demonstrate that, in static cultures, oxygen diffusion into the medium is rate limiting and that cells experience widely varying oxygen levels as a result. This is not a surprising result if one considers that the diffusion coefficient for oxygen in nitrogen is 0.240 cm²s⁻¹, but for oxygen in water it is only 0.0000324 cm²s⁻¹(56). Therefore, a strategy for gently agitating the culture vessel to promote better mixing should result in better equilibration of the gas and liquid phases and promote more uniform oxygen levels.

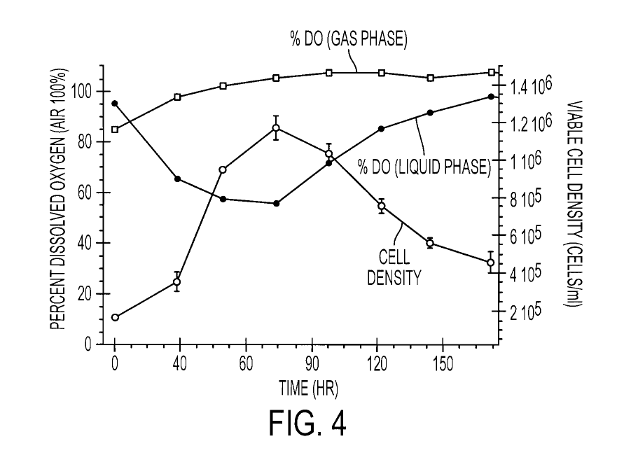

This approach was tested in the following experiment. An aquarium pump was placed on the top shelf of the CO₂ incubator and turned on for the duration of the experiment. The steady vibrations emitted by the pump were sufficient to gently mix the liquid medium and allowed for much greater oxygen diffusion from the headspace into the liquid. As seen in the plot of FIG. 4, and in contrast with the static culture results shown in FIG. 3, the liquid phase oxygen levels were observed to be above 50% throughout the duration of the culture.

The vibration intensity was constant, however, had we been able to control its intensity, we should have been able to obtain a different oxygen profile in the liquid. These data provide proof-of-principle that agitation can be used to provide feedback control of pO₂ levels in cell culture vessels in an incubator.

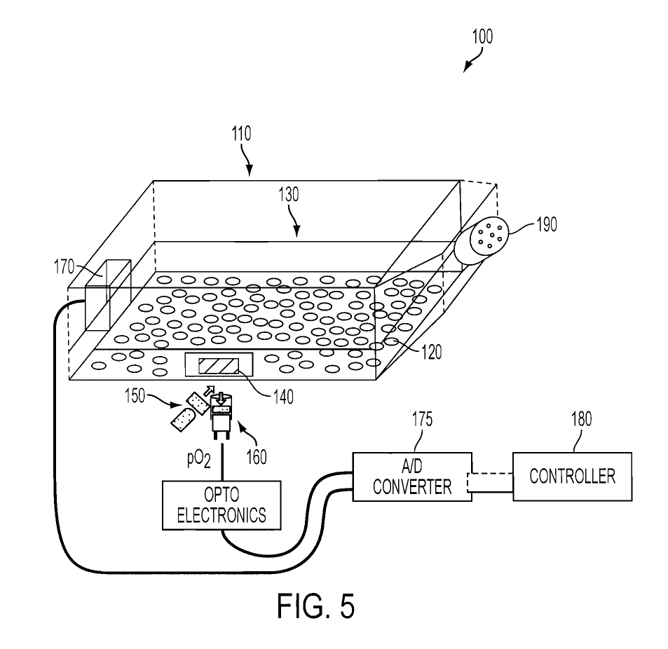

FIG. 5 is a schematic diagram of a system 100 for non-invasive measurement and control of pO₂ in a culture vessel, in accordance with one embodiment of the present invention. The system 100 is designed for use in an incubator (not shown).

The system includes a culture vessel 110 that holds cells 120 and a cell culture medium 130. An oxygen (pO₂) sensor patch 140 is affixed inside the culture vessel 110, and is monitored optically (non-invasively) from the outside using an illuminating light source 150 and a photodetector 160 for measuring the optical signal emitted by the oxygen sensor patch 140. An agitator, suitably a vibratory mixer 170, is attached to the culture vessel 110 for providing agitation and promote rapid equilibration between the culture medium 130 and the gas phase. The vibratory mixer 170 is preferably under the control of a controller 180, such as a computer. pO₂ control of gas phase concentrations is controlled by the controller 180. The mixer driven equilibration will result in precise control of the liquid medium pO₂.

The culture vessel 110 is suitably a T-flask with a 0.2 m pore size filter vented cap 190. These minimize diffusive resistance between the C-chamber atmosphere and the headspace in the flask and provide a tight and sterile seal. However, the culture vessel 110 can be any type of vessel that can be used to culture cells such as, for example, a Petri dish, a spin tube, a spinner flask or a shaker flask. The oxygen sensor 140 is not affected by the size of vessel 110 used.

The oxygen sensor patch 140 contains an oxygen-sensitive luminescent dye that is a complex of Ruthenium, Platinum, or any other metal ligand complex. The compound is preferably immobilized in silicone rubber to provide an inert, steam, ethanol or radiation sterilizable patch. The oxygen sensor patch 140 is then calibrated. One advantage of this type of oxygen sensor patch 140 is that calibration is required on only one of the patches when a batch of patches is made. All patches from a common lot will behave identically, so individual calibration of each patch is not required. The measurement of the dissolved oxygen is preferably based on Stern-Volmer quenching of emission, as described below.

The excited state of the luminescent dye in the oxygen sensor patch 140 is quenched proportionately to the oxygen concentrations. Intensity-modulated light at a frequency (w=2p×Hz) generated from the light source 150, preferably light emitting diodes (LED's), serves as the excitation source. The phase shift (f) of the resulting emission from the oxygen sensor patch 140 is related to the decay time (t) by

tan f_{w}=wt (1)

Collisional quenching by oxygen is described by the Stern-Volmer equation,

t₀/t=tan f₀/tan f=1+Ksv[Q] (2)

where t₀ and f₀ are the decay time and phase in the absence of oxygen, respectively, Ksv is the Stern-Volmer constant and is the oxygen concentration.

For calibration, the phase angle is measured as a function of various mixtures of oxygen in nitrogen and a curve fit is performed. This is then entered into software on the controller 180 and, when an oxygen measurement is needed, the software automatically calculates the oxygen concentration from the calibration file. The typical calibration curve behaves like a saturation binding curve. Consequently, the oxygen sensor patch 140 becomes more sensitive as the oxygen tension decreases and has greater accuracy at lower oxygen levels.

The oxygen sensor patch 140 is preferably steam sterilized and inserted into the culture vessel 110 under sterile operating conditions in a laminar flow hood. The oxygen sensor patch 140 preferably have a biocompatible adhesive that is designed as a “peel-and-stick” unit.

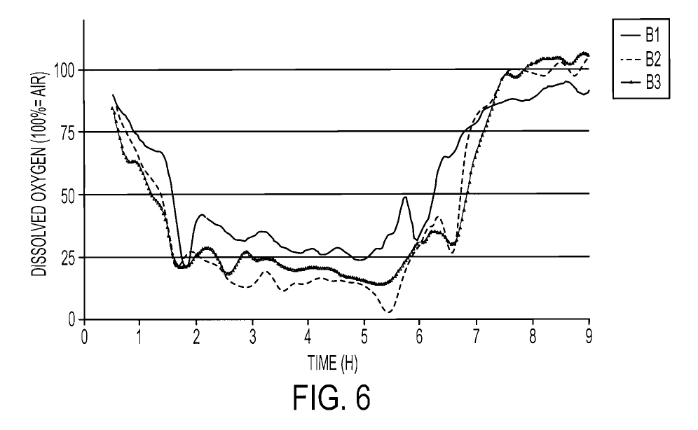

The accuracy of the preferred oxygen sensor patch described above has been extensively valiated (45,47,51,52,55). FIG. 6 is a plot comparing the preferred oxygen sensor patch 140 with conventional electrochemical sensors (a Clark electrode) during a cell culture experiment (45). As the data show, the optical sensor data agree well with the Clark oxygen electrode. Furthermore, the preferred oxygen sensor patch 140 has been extensively validated using microarray analysis to ensure that the sensor patch chemistry and/or optical energy used for the measurement do not affect the cells or product adversely (47).

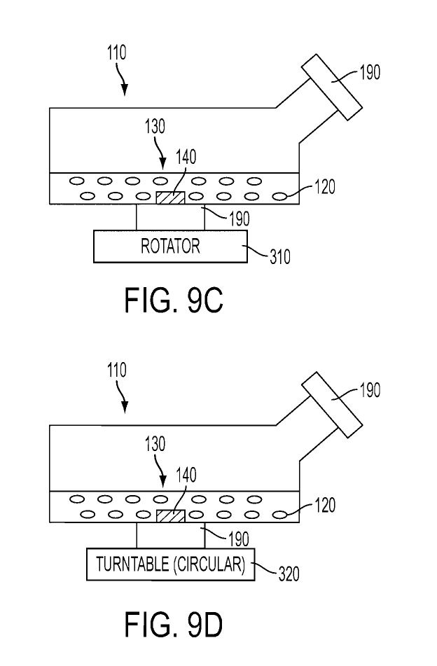

The plot of FIG. 9 also demonstrates pO₂ control in a 24 well plate system where 3 wells were equipped with oxygen sensor patches 140 and computer control was set at a setpoint of 20% (55). The cells were E. coli, which have a very high oxygen demand compared to mammalian cells and are much harder to control.

Initially, the oxygen levels were high until the cells grew to a point where oxygen supply was less than the consumption rate of the respiring cells. Once the oxygen levels reached the setpoint, control was achieved by increasing the stirring speed in the wells whenever oxygen dropped below the setpoint. The increased mixing had the effect of increased oxygen transport into the liquid phase and raised its level. If the oxygen level crossed the setpoint, then the stirring speed was decreased. This control algorithm relies on cells consuming the oxygen to maintain a steady oxygen level and was done by feedback control using the controller, preferably a computer.

Towards the end (six hours onwards) after the cells ran out of nutrient and respiration slowed down, the oxygen levels again rose and reached equilibrium with the supplied air. Reasonable accuracy was obtained despite no attempt being made to tune the control algorithm.

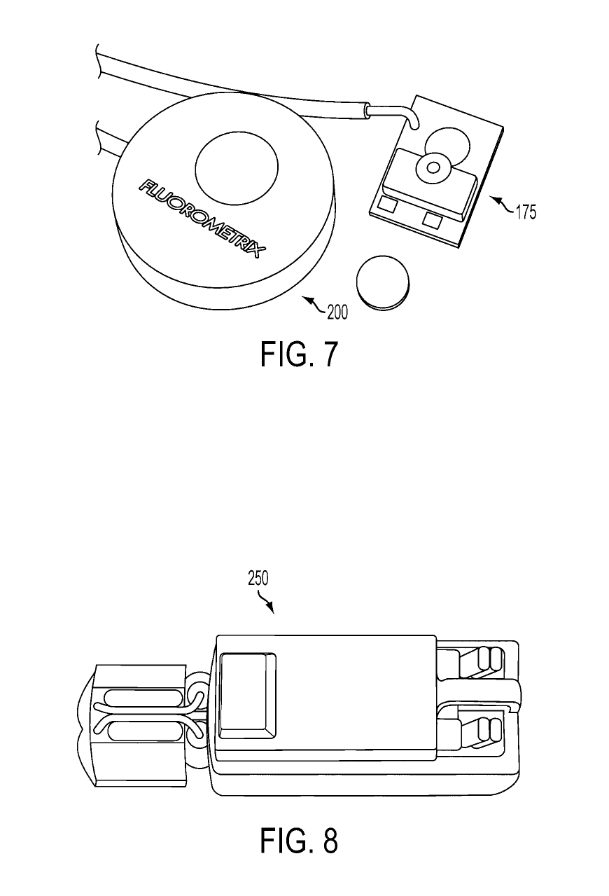

The light source 150 and the photodetector 140 are preferably packaged together to form an oxygen sensor module 200, as shown in the photograph of FIG. 7. The oxygen sensor module 200 is mounted such that the light from the light source 150 illuminates the oxygen sensor patch 140. The emission from the oxygen sensor patch 140 is then detected by photodetector 160. The oxygen sensor module 200 is preferably connected to an analog-to-digital converter (ADC) 175, that communicates with the controller 180. The ADC 175 preferably has a variable voltage output that is used to drive the vibratory mixer 170 under the control of the controller 180.

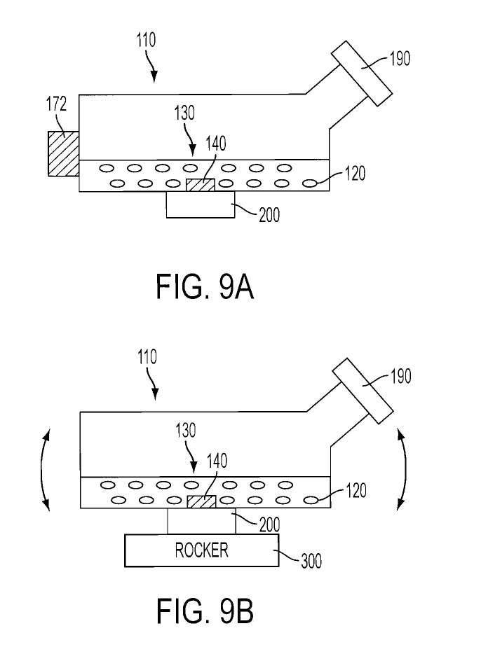

The vibratory mixer 170 is preferably a miniature vibration motor 250, such as the one shown in the photograph of FIG. 8, that is typically used in cell phones and pagers. This type of vibrator motor body is typically only 0.44″ L×0.18″ Dia., and has a weight attached to its shaft to provide inertial damping. It has 2 flexible terminals (an important feature for a vibrating device so that the power leads do not fall off) for power connection and can operate from 1VDC up to 9VDC. The resistance of the motor is 10 ohms.

The vibration motor 250 is preferably affixed to the culture vessel 110 with double-sided tape. The optimum location on the culture vessel 110 to attach the vibration motor 250 is experimentally determined based on simple mixing studies conducted by dropping a colored dye into water and observing the mixing patterns, as has been done with minibioreactors (58). This allows one to choose the vibration motor location that results in the shortest mixing times. It is important to avoid a location that matches the natural resonant frequency of the system, as that will result in standing waves and poor mixing.

Although the agitator shown in FIG. 5 is a vibratory mixer 170, any other type of agitator known in the art may be used while still falling within the scope of the present invention. FIGS. 9A-9D illustrate examples of other types of agitators that may be used. FIG. 9A shows the use of an ultrasonic transducer 172 to induce ultrasonic waves in the culture medium 130. FIG. 9B illustrates the use of a rocker 300. The oxygen sensor module 200 is attached to the bottom of the culture vessel 110, and the culture vessel/oxygen sensor module combination sits on top of the rocker 300. The rocker 300 tilts the culture vessel/oxygen sensor module combination back and forth in a “teeter-totter” motion to agitate the culture medium 130.

FIG. 9C illustrates the use of a rotator 310 to agitate the culture medium 130. The culture vessel/oxygen sensor module combination sits on top of the rotator, which gyrates in a back-and-forth circular motion.

FIG. 9D illustrates the use of a turntable 320 to agitate the culture medium 130. The culture vessel/oxygen sensor module combination sits on top of the turntable, which rotates to swirl the culture vessel/oxygen sensor module combination.

The agitator, whichever type is used, is preferably powered with a feedback loop from the sensor software run by the controller 180 to agitate when oxygen levels drop below a user entered set point. When this happens, the diffusion of oxygen into the liquid phase will increase until the set point is reached, at which point the controller will turn the motor off.

All of the instrumentation is preferably contained in water-proof enclosures. This allows them to be sprayed with 70% ethanol and wiped down prior to placement in the incubator chamber to maintain sterility. It also prevents fogging of the optical windows internally during use in a humidified incubator.

The controller suitably runs a LabVIEW based data acquisition software. When a vibratory mixer 170 is used as the agitator, dissolved oxygen is controlled by changing the vibration rate of the vibratory mixer 170. The vibration rate is changed by controlling the voltage supply to the vibratory mixer 170. pO₂ is preferably controlled by the following well-known PID (proportional-integral-derivative) algorithm for sampled systems:

$${A}_{n} = {A}_{n - 1} + {K}_{c} [ {E}_{n} ( 1 + \frac{T}{{τ}_{i}} + \frac{{τ}_{d}}{T} ) - {E}_{n - 1} ( 1 + \frac{2 {τ}_{d}}{T} ) + {E}_{n - 2} \frac{{τ}_{d}}{T} ] , \qquad ( 3 )$$

where A is the controller output (vibration rate), E is the difference between the measured and desired pO_{2,} and K_{c}, t, and t_{d} are the gain, integral time constant, and derivative time constant, respectively. T is the time between successive measurements.

The design of the control software assumes that cells are actively consuming oxygen. Control is achieved by varying the oxygen supply rate, which is a function of the agitation rate provided by the vibratory mixer 170. This is significant, as it means that the upper limit for control will be determined by the gas phase oxygen concentration. At equilibrium, this will be the maximum pO₂ achievable in the liquid. The lower ranges will be determined by the agitation rate. In practice, for studies on normoxic or hypoxic respiring cells, we hypothesize that it may be possible to eliminate expensive gas phase environment controls. With respiring cells, one could simply run all incubators with inexpensive house air and achieve control at any desired oxygen level with the systems and methods of the present invention. This could be achieved at a fraction of the cost of an incubator controller.

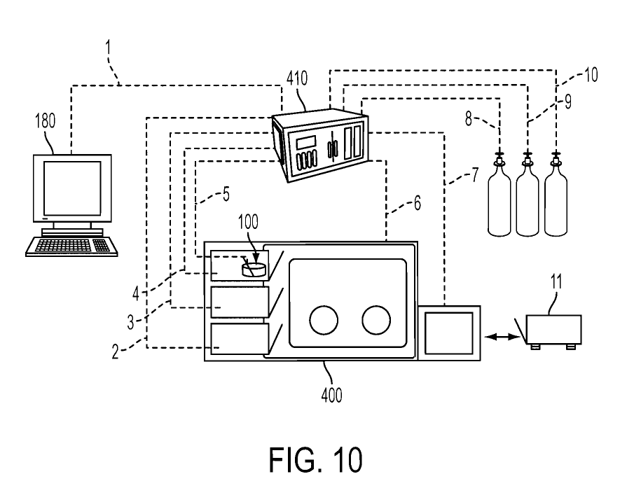

As discussed above, the system 100 is designed to be placed inside any type of incubator. FIG. 10 schematically shows an incubator 400 that is supplied with a blend of O₂, N₂ and CO₂ in any desired combination using a commercially available oxygen controller 410, such as the OxyCycler from Biospherix. The system 100 would preferably placed inside a cell culture C-Chamber that resides inside the temperature controlled incubator 400.

The foregoing embodiments and advantages are merely exemplary, and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art. Various changes may be made without departing from the spirit and scope of the invention, as defined in the following claims (after the Appendix below).

### APPENDIX

1. Jones, P. H., S. Harper, and F. M. Watt, Stem cell patterning and fate in human epidermis. Cell, 1995.80(1): p. 83-93;
2. Fuchs, E. and J. A. Segre, Stem cells: a new lease on life. Cell, 2000. 100(1): p. 143-55;
3. Gawronska-Kozak, B., Regeneration in the ears of immunodeficient mice: identification and lineage analysis of mesenchymal stem cells. Tissue Eng, 2004. 10(7-8): p. 1251-65;
4. Gage, F. H., J. Ray, and L. J. Fisher, Isolation, characterization, and use of stem cells from the CNS. Annu Rev Neurosci, 1995.18: p. 159-92;
5. Lechner, A. and J. F. Habener, Stem/progenitor cells derived from adult tissues: potential for the treatment of diabetes mellitus. Am J Physiol Endocrinol Metab, 2003. 284(2): p. E259-66;
6. Jankowski, R. J., B. M. Deasy, and J. Huard, Muscle-derived stem cells. Gene Ther, 2002. 9(10): p. 642-7;
7. Cao, B., et al., Muscle stem cells differentiate into haematopoietic lineages but retain myogenic potential. Nat Cell Biol, 2003.5(7): p. 640-6;
8. Gimble, J. M. and F. Guilak, Differentiation potential of adipose derived adult stem (ASC) cells. CurrTop Dev Biol. 2003.58: p. 137-60;
9. Spradling, A., Drummond-Barbosa, D., and Kai, T. 2001. Stem cells find their niche. Nature 414: 98-104;
10. D. L. Jones. Stem Cells: So what's in a niche? (2001). Curr. Biol. 11: R484-R486;
11. Scadden, D. T. The stem-cell niche as an entity of action. 2006. Nature 441:1075-1079;
12. Qun Lin, Yi-Jang Lee, and Zhong Yun Differentiation Arrest by Hypoxia J. Biol. Chem. 2006 281:30678-30683;
13. Diana L. Ramirez-Bergeron and M. Celeste Simon. Hypoxia-Inducible Factor and the Development of Stem Cells of the Cardiovascular System. 2001;19;279-286 Stem Cells;
14. Hevehan D L, Papoutsakis E T, Miller W M. Physiologically significant effects of pH and oxygen tension on granulopoiesis. Experimental Hematology. 2000; 28(3):267-75;
15. Koller M R, Bender J G, Miller W M, and Papoutsakis E T (1992). Reduced oxygen tension increases hematopoiesis in long-term culture of human stem and progenitor cells from cord blood and bone marrow. Exp. Hematol. 20(2):264-270;
16. Kondo, M., et al., Biology of hematopoietic stems cells and progenitors: implications for clinical application. Annu Rev Immunol, 2003.21: p. 759-806.;
17. Caplan, A. I., Mesenchymal stem cells. J Orthop Res, 1991. 9(5): p. 641-50;
18. Friedenstein, A. J., Precursor cells of mechanocytes. Int Rev Cytol, 1976. 47: p. 327-59;
19. Gimble, J. M. and M. E. Nuttall, Bone and fat: old questions, new insights. Endocrine. 2004.23(2-3): p. 183-8;
20. Jiang, Y., et al., Multipotent progenitor cells can be isolated from postnatal murine bone marrow, muscle, and brain. Exp Hematol, 2002. 30(8): p. 896-904;
21. Ferraris, C, et al., Adult corneal epithelium basal cells possess the capacity to activate epidermal, pilosebaceous and sweat gland genetic programs in response to embryonic dermal stimuli. Development, 2000.127(24): p. 5487-95;
22. Fraser, J. K., Wulur, I. Zeni, A. and Hedrick, M. H., Fat Tissue: an underappreciated source of stem cells for biotechnology, Trends Biotechnol. 2006April; 24(4):150-4;
23. Gimble, J. M. and F. Guilak, Differentiation potential of adipose derived adult stem (ASC) cells. CurrTop Dev Biol, 2003. 58: p. 137-60;
24. Rodbell, M., Metabolism of isolated fat cells. II. The similar effects of phospholipase C (Clostridium perfringens alpha toxin) and of insulin on glucose and amino acid metabolism. J Biol Chem, 1966.241(1): p. 130-9;
25. Rodbell, M., The metabolism of isolated fat cells. IV. Regulation of release of protein by lipolytic hormones and insulin. J Biol Chem, 1966. 241(17): p. 3909-17;
26. Rodbell, M. and A. B. Jones, Metabolism of isolated fat cells. 3. The similar inhibitory action of phospholipase C (Clostridium perfringens alpha toxin) and of insulin on lipolysis stimulated by lipolytic hormones and theophylline. J Biol Chem, 1966. 241(1): p. 140-2;
27. Hauner, H., et al., Promoting effect of glucocorticoids on the differentiation of human adipocyte precursor cells cultured in a chemically defined medium. J Clin Invest, 1989. 84(5): p. 1663-70;
28. Daniel J Ceradini, Anita R Kulkarni, Matthew J Callaghan, Oren M Tepper, Nicholas Bastidas, Mark E Kleinman, Jennifer M Capla, Robert D Galiano, Jamie P Levine & Geoffrey C Gurtner. Progenitor cell trafficking is regulated by hypoxic gradients through HIF-1 induction of SDF-1 Nature Medicine 10, 858 - 864 (2004);
29. Katz, A. J., et al., Cell Surface and Transcriptional Characterization of Human Adipose-Derived Adherent Stromal (hASC) Cells. Stem Cells, 2005. 23(3): p. 412-23
30. Miranville, A., et al., Improvement of postnatal neovascularization by human adipose tissue-derived stem cells. Circulation, 2004.110(3): p. 349-55;
31. Kevin McIntosh, Sanjin Zvonic, Sara Garrett, James B. Mitchell, Z Elizabeth Floyd, Lora Hammill, Amy Kloster, Yuan Di Halvorsen, Jenny P. Ting, Robert W. Storms, Brian Goh, Gail Kilroy, Xiying Wu and Jeffrey M. Gimble. The Immunogenicity of Human Adipose-Derived Cells: Temporal Changes In Vitro. Stem Cells. 2006; 24:1246-1253;
32. James B. Mitchell, Kevin McIntosh, Sanjin Zvonic, Sara Garrett, Z. Elizabeth Floyd, Amy Kloster, Yuan Di Halvorsen, Robert W. Storms, Brian Goh, Gail Kilroy, Xiying Wu and Jeffrey M. Gimble Immunophenotype of Human Adipose-Derived Cells: Temporal Changes in Stromal-Associated and Stem Cell-Associated Markers Stem Cells 2006; 24;376-385.;
33. Halliwell, B. and Gutteridge, J. Free Radicals in Biology and Medicine. 2007. Oxford University Press;
34. Semenza, G. L. Perspectives on oxygen sensing. Cell. 1999.98:281-284;
35. Semenza, G. L. HIF-1 and human disease: one highly involved factor 2000 14:1983-1991 Genes & Dev;
36. Reya T, Morrison S J, Clarke M F, Weissman I L. Stem cells, cancer, and cancer stem cells. Nature. 2001 Nov. 1; 414(6859): 105-11;
37. Malladi, P. Yue Xu, Michael Chiou, Amato J. Giaccia, and Michael T. Longaker. Effect of reduced oxygen tension on chondrogenesis and osteogenesis in adipose-derived mesenchymal cells Am J Physiol Cell Physiol 290: C1139- C1146, 2006;
38. Falanga V, and Kirsner R S. (1993). Low oxygen stimulates proliferation of fibroblasts seeded as single cells. J Cell Physiol. 154(3): 506-510;
39. Reykdal S. Abboud C, and Liesveld J (1999). Effect of nitric oxide production and oxygen tension on progenitor preservation in ex vivo culture. Exp. Hematol. 27(3):441-450;
40. Annabi B. Lee Y T, Turcotte S, Naud E, Desrosiers R R. Champagne M, Eliopoulos N. Galipeau J, Beliveau R. (2003). Hypoxia promotes murine bone-marrow derived stromal cell migration and tube formation. Stem Cells. 21(3):337-347;
41. Studer L, Csete M. Lee S. Kabbani N, Walikonis J. Wold B and McKay R. (2000). Enhanced proliferation, survival and Dopaminergic differentiation of CNS precursors in lowered oxygen. J of Neurosci. 20(19):7377-7383;
42. Wang. D. W., Fermor, B. Gimble, J. M., Awad, H. A., Guilak, F. Influence of Oxygen on the Proliferation and Metabolism of Adipose Derived Adult Stem Cells. J. Cell. Phys. 204:184-191 (2005)
43. Pittenger, M. F., Alastair M. Mackay, Stephen C. Beck, Rama K. Jaiswal, Robin Douglas, Joseph D. Mosca, Mark A. Moorman, Donald W. Simonetti, Stewart Craig, Daniel R. Marshak Multilineage potential of adult human mesenchymal stem cells. Science, 1999.284(5411): p. 143-7;
44. Randers-Eichhorn, L., Roscoe Bartlett, Douglas Frey and Govind Rao. Non-Invasive Oxygen Measurements and Mass Transfer Limitations in Tissue Culture Flasks. Biotechnol. Bioeng. 1996. 51:466-478;
45. Michael A. Hanson, Xudong Ge, Yordan Kostov, Kurt A. Brorson, Antonio R. Moreira, Govind Rao Comparisons of optical pH and Dissolved Oxygen sensors with traditional electrochemical probes during mammalian cell culture. Biotechnol. Bioeng. 2007. 97:833-841;
46. Ge, X., Yordan Kostov and Govind Rao. High-Stability Non-Invasive Autoclavable Naked Optical C02 Sensor. 2003. Biosensors and Bioelectronics. 18:857-865.
47. Ge X. Hanson M, Shen H, Kostov Y, Brorson K A, Frey D D, Moreira A R, Rao G. Validation of an optical sensor-based high-throughput bioreactor system for mammalian cell culture. J Biotechnol. 2006.122:293-306;
48. Kostov, Y., Van Houten, K. A., Harms. P., Pilato, R. S., Rao, G. 2000a. A Unique Oxygen Analyzer Combining a Dual Emission Probe and a Low-Cost Solid-State Ratiometric Fluorometer. Appl. Spectroscopy 54:864-868;
49. Kostov, Y., Harms, P., Pilato, R. S. and Rao, G. 2000b. Ratiometric oxygen sensing: detection of dual-emission ratio through a single emission filter. Analyst 125:1175-1178;
50. Van Houten, K. A., Heath, D. C, Barringer, C. A., Rheingold, A. L. and Pilato, R. S. 1998. Functionalized 2-pyridyl-substituted metallo-1,2-enedithiolates. Synthesis, characterization, and photophysical properties of (dppe)M{S₂C₂(2-pyridine(ium))(CH₂CH₂OR″)} and (dppe)M[{S₂C₂(CH₂CH₂—N-2-pyridinium)}]⁺ (R″═H, Acetyl, Lauroyl; M=Pd, Pt; dppe=1,2-Bis(diphenylphosphino)ethane). Inorg. Chem. 37:4647;
51. Atul Gupta and Govind Rao. A Study Of Oxygen Transfer In Shake Flasks Using A Non-Invasive Oxygen Sensor. 2003. Biotechnol. Bioeng. 84:351-358;
52. Bambot S. B., Holavanhali R. Lakowicz J. R., Carter G. M., Rao G. 1994. Phase fluorimetric sterilizable optical oxygen sensor. Biotechnol Bioeng 43:1139-1145;
53. Kermis, H. R., Kostov, Y., Rao, G. Rapid method for the preparation of a robust optical pH sensor. 2003. Analyst. 128:1181-1186;
54. Xudong Ge, Yordan Kostov and Govind Rao. Low-cost noninvasive C02 sensing system for fermentation and cell culture. 2005. Biotechnol. Bioeng. 89:329-334;
55. Peter Harms, Yordan Kostov, Joseph A. French, Mohammed Soliman, M. Anjanappa, Arun Ram, Govind Rao. Design and Performance of a 24 Station High Throughput Microbioreactor. 2006. Biotechnol. Bioeng. 93:6-13;
56. Welty, J. R., C. E. Wicks, and R. E. Wilson. 1984. Fundamentals of Momentum, Heat, and Mass Transfer, Third Edition. John Wiley & Sons.NY, p. 803;
57. Govind Rao, Marco Cacciuttolo and Cynthia Oliver. Animal Cell Culture: Physiochemical Effects of Dissolved Oxygen and Redox Potential. Encyclopedia of Cell Culture Technology. 2000 John Wiley and Sons, NY. pp 51-57;
58. Jose Vallejos, Yordan Kostov. Arun Ram, Joseph A. French. Mark Marten and Govind Rao, Optical Analysis of Liquid Mixing in a Minibioreactor. 2006. Biotechnol. Bioeng. 93:906-911;
59. Ge, X., Yordan Kostov and Govind Rao, High-Stability Non-Invasive Autoclavable Naked Optical CO2 Sensor. 2003. Biosensors and Bioelectronics. 18:857-865; and
60. Xudong Ge, Yordan Kostov and Govind Rao, Low-cost noninvasive C02 sensing system for fermentation and cell culture. 2005. Biotechnol. Bioeng. 89:329-334.
61. Jacem Kilani and Jean-Michel Lebeault, Study of the oxygen transfer in a disposable flexible bioreactor with surface aeration in vibrated medium, 2007, Appl. Microbio. Biotechnol, 74:324-330.

What is claimed is:

1. A system, comprising:
a culture vessel for holding a culture medium;
an oxygen sensor positioned inside the culture vessel for detecting dissolved oxygen in the culture medium, wherein the oxygen sensor is adapted to be monitored non-invasively;
an agitator attached to the culture vessel for providing agitation to the culture medium; and
a controller for determining a dissolved oxygen content of the culture medium based on data from the oxygen sensor and for controlling the agitator based on the dissolved oxygen content of the culture medium.

2. The system of claim 1, wherein the oxygen sensor comprises an optical oxygen sensor.

3. The system of claim 2, wherein the optical oxygen sensor comprises an oxygen sensitive luminescent dye.

4. The system of claim 3, further comprising:
a light source positioned outside the culture vessel for illuminating the optical oxygen sensor; and
a photodetector positioned outside the culture vessel for detecting an optical signal from the optical oxygen sensor.

5. The system of claim 3, wherein the oxygen sensitive luminescent dye is immobilized in silicone rubber.

6. The system of claim 5, wherein the optical oxygen sensor is attached to an inside wall of the culture vessel with a biocompatible adhesive.

7. The system of claim 1, wherein the culture vessel comprises a T-flask.

8. The system of claim 1, wherein the controller turns on the agitator when a dissolved oxygen level in the culture medium falls below a predetermined set point.

9. The system of claim 1, wherein the controller controls the agitator so as to maintain equilibrium between oxygen gas in the culture vessel and dissolved oxygen in the culture medium.

10. The system of claim 1, wherein the agitator comprises a vibrator motor.

11. The system of claim 1, wherein the agitator comprises an ultrasonic transducer.

12. The system of claim 1, wherein the agitator comprises rocker.

13. The system of claim 1, wherein the agitator comprises a rotator.

14. The system of claim 1, wherein the agitator comprises a turntable.

15. A system, comprising:
an incubator;
at least two culture vessels positioned inside the incubator for holding respective culture media;
an oxygen sensor positioned in each of the at least two culture vessels for detecting dissolved oxygen in each culture vessel, wherein the oxygen sensors are adapted to be monitored non-invasively;
an agitator attached to each of the at least two culture vessels for providing agitation to each culture medium; and
a controller for determining a dissolved oxygen content of the culture medium in each culture vessel based on data from the oxygen sensors and for independently controlling each agitator based on the dissolved oxygen content of the respective culture medium.

16. The system of claim 15, wherein each oxygen sensor comprises an optical oxygen sensor.

17. The system of claim 16, wherein each optical oxygen sensor comprises an oxygen sensitive luminescent dye.

18. The system of claim 17, further comprising:
a light source positioned outside each culture vessel for illuminating a respective optical oxygen sensor; and
a photodetector positioned outside each culture vessel for detecting an optical signal from the respective optical oxygen sensor.

19. The system of claim 17, wherein the oxygen sensitive luminescent dye is immobilized in silicone rubber.

20. The system of claim 19, wherein each optical oxygen sensor is attached to an inside wall of a respective culture vessel with a biocompatible adhesive.

21. The system of claim 15, wherein each culture vessel comprises a T-flask.

22. The system of claim 15, wherein the controller selectively turns on an agitator when a dissolved oxygen level in the respective culture medium falls below a predetermined set point.

23. The system of claim 15, wherein the controller selectively and independently controls each agitator.

24. The system of claim 23, wherein the controller selectively and independently controls each agitator so as to maintain equilibrium between oxygen gas in the respective culture vessel and dissolved oxygen in the respective culture medium.

25. The system of claim 15, wherein each agitator comprises a vibrator motor.

26. The system of claim 15, wherein the agitator comprises an ultrasonic transducer.

27. The system of claim 15, wherein the agitator comprises rocker.

28. The system of claim 15, wherein the agitator comprises a rotator.

29. The system of claim 15, wherein the agitator comprises a turntable.

30. A method, comprising:
non-invasively monitoring dissolved oxygen levels in at least two culture media; and
selectively and independently agitating the at least two culture media based on the dissolved oxygen levels in each culture medium.

31. The method of claim 30, wherein the at least two culture media are independently agitated by vibrating the culture media.

32. The method of claim 30, wherein the dissolved oxygen level in each culture medium is non-invasively monitored with optical oxygen sensors positioned in respective culture vessels that hold the at least two culture media.

33. The method of claim 31, wherein the at least two culture media are vibrated with respective vibration motors attached to respective culture vessels that hold the at least two culture media.

34. The method of claim 30, wherein each culture medium is selectively and independently agitated so as to maintain equilibrium between oxygen gas in contact with each culture medium and dissolved oxygen in each culture medium.

35. The method of claim 30, wherein the dissolved oxygen levels in each culture medium are independently monitored by monitoring fluorescence signals from respective optical oxygen sensors.

36. A method, comprising:
non-invasively monitoring dissolved oxygen levels in a culture medium; and
selectively agitating the culture medium based on the dissolved oxygen level in each culture medium.

37. The method of claim 36, wherein the culture medium is selectively agitated by vibrating the culture medium.

38. The method of claim 36, wherein the culture medium is selectively agitated so as to maintain equilibrium between oxygen gas in contact with the culture medium and dissolved oxygen in the culture medium.
